(19) United States
(12) Patent Application Publication
(10) Pub. No.: US 2015/0036792 A1
(43) Pub. Date: Feb. 5, 2015

(54) COMPUTED TOMOGRAPHY APPARATUS, AND METHOD OF GENERATING IMAGE BY USING COMPUTED TOMOGRAPHY APPARATUS

(71) Applicants: SAMSUNG ELECTRONICS CO., LTD., Suwon-si (KR); KOREA ADVANCED INSTITUTE OF SCIENCE AND TECHNOLOGY, Daejeon (KR)

(72) Inventors: Jonghyon YI, Yongin-si (KR); Seung-ryong CHO, Daejeon (KR); Toshihiro RIFU, Suwon-si (KR); Dae-hyung PARK, Yongin-si (KR); Tae-won LEE, Daejeon (KR)

(73) Assignees: KOREA ADVANCED INSTITUTE OF SCIENCE AND TECHNOLOGY, Daejeon (KR); SAMSUNG ELECTRONICS CO., LTD., Suwon-si (KR)

(21) Appl. No.: 14/449,587

(22) Filed: Aug. 1, 2014

Related U.S. Application Data
(60) Provisional application No. 61/861,026, filed on Aug. 1, 2013.

(30) Foreign Application Priority Data
Sep. 16, 2013 (KR) 10-2013-0111183

(51) Int. Cl. A61B 6/00; A61B 6/03; A61B 6/06

(52) U.S. Cl. CPC A61B 6/542; A61B 6/06; A61B 6/482; A61B 6/035
USPC 378/4

(57) ABSTRACT

A computed tomography apparatus includes an X-ray irradiation unit irradiating an X-ray to an object while rotating along a predetermined rotation path, a detector acquiring projection data by detecting an X-ray transmitted through the object, a filter unit located between the X-ray irradiation unit and the object and comprising a plurality of transmissive areas and a plurality of slightly transmissive areas that are arranged in a predetermined direction, and a control unit controlling a motion of the filter unit to allow a relative position of the plurality of transmissive areas with respect to the X-ray irradiation unit and a relative position of the plurality of slightly transmissive areas with respect to the X-ray irradiation unit to change while the X-ray irradiation unit rotates.

### RELATED APPLICATIONS

This application claims the benefit of U.S. Provisional Patent Application No. 61/861,026, filed on Aug. 1, 2013, in the USPTO, and Korean Patent Application No. 10-2013-0111183, filed on Sep. 16, 2013, in the Korean Intellectual Property Office, the disclosures of which are incorporated herein in their entirety by reference.

### BACKGROUND

1. Field

One or more exemplary embodiments relate to a medical imaging apparatus, and more particularly, to a computed tomography (CT) apparatus and a method of generating an image by using a CT apparatus.

2. Description of the Related Art

Medical imaging apparatuses acquire images of an internal structure of an object. A medical imaging apparatus is a non-invasive diagnostic apparatus that shows structural details, internal tissue, and a flow of a liquid in a human body. A medical imagining apparatus may be a magnetic resonance imaging (MRI) apparatus, a computed tomography (CT) apparatus, an X-ray apparatus, an ultrasound apparatus, etc.

Among the medical imaging apparatuses, a CT apparatus may provide a sectional image of an object and may present the internal structure of an object, for example, an organ such as a liver, a lung, etc. without overlapping, compared to a general X-ray apparatus.

However, the X-ray irradiated by the CT apparatus may increase the amount of radiation exposure to a patient. In particular, for pregnant women, the X-ray of a CT apparatus may cause a serious disease and/or develop a complication and may have a critical influence on the growth and development of a fetus. Accordingly, an efficient and safe method to reduce the amount of X-ray radiation of a CT apparatus is needed.

### SUMMARY

One or more exemplary embodiments include a computed tomography (CT) apparatus and a method of generating an image by using a CT apparatus which may reduce an amount of radiation of an X-ray to an object by using a general CT apparatus.

One or more exemplary embodiments include a CT apparatus and a method of generating an image by using a CT apparatus which may scan using dual energy with a low dose radiation by using a general CT apparatus.

Additional aspects will be set forth in part in the description which follows and, in part, will be apparent from the description, or may be learned by practice of the presented exemplary embodiments.

According to one or more exemplary embodiments, a computed tomography apparatus includes an X-ray irradiation unit irradiating an X-ray to an object while rotating along a predetermined rotation path, a detector acquiring projection data by detecting an X-ray transmitted through the object, a filter unit located between the X-ray irradiation unit and the object and comprising a plurality of transmissive areas and a plurality of slightly transmissive areas that are arranged in a predetermined direction, and a control unit controlling a motion of the filter unit to allow a relative position of the plurality of transmissive areas with respect to the X-ray irradiation unit and a relative position of the plurality of slightly transmissive areas with respect to the X-ray irradiation unit to change while the X-ray irradiation unit rotates.

The plurality of transmissive areas and the plurality of slightly transmissive areas may be alternatingly arranged in the predetermined direction.

An X-ray attenuation rate of the plurality of transmissive areas may be lower than an X-ray attenuation rate of the plurality of slightly transmissive areas.

The X-ray attenuation rate of the plurality of transmissive areas may be 50% to 95% of the X-ray attenuation rate of the plurality of slightly transmissive areas.

The total size of the plurality of transmissive areas may be smaller than the total size of the plurality of slightly transmissive areas.

The total size of the plurality of transmissive areas may be smaller than ½ of the total size of the plurality of slightly transmissive areas.

The filter unit may include a flat panel in which the plurality of transmissive areas and the plurality of slightly transmissive areas may be formed in a direction perpendicular to a rotational axis of the X-ray irradiation unit.

The control unit may control a motion of the flat panel to allow the flat panel to perform a reciprocal motion in a direction in which the plurality of transmissive areas and the plurality of slightly transmissive areas are formed.

The control unit may control a motion of the flat panel to allow the flat panel to reciprocate 10 to 100 times while the X-ray irradiation unit rotates one time.

The filter unit may include an air compressor motor or a vibration motor that transmits a drive force for the reciprocal motion to the flat panel.

The filter unit may include a flat panel in which the plurality of transmissive areas and the plurality of slightly transmissive areas are formed in a circular direction.

The control unit may control a motion of the flat panel to allow the flat panel to rotate in the circular direction.

The control unit may control a motion of the flat panel to allow the flat panel to perform a rotational motion one to five times while the X-ray irradiation unit rotates one time.

The filter unit may include a caterpillar panel in which the plurality of transmissive areas and the plurality of slightly transmissive areas are formed in a direction perpendicular to a rotational axis of the X-ray irradiation unit, and a plurality of driving rollers contacting an inner circumference of the caterpillar panel.

The control unit may control motions of the plurality of driving rollers to allow the caterpillar panel to move in a direction in which the plurality of transmissive areas and the plurality of slightly transmissive areas are formed.

The detector may acquire first projection data by detecting an X-ray transmitted through the plurality of transmissive areas and second projection data by detecting an X-ray transmitted through the plurality of slightly transmissive areas, and the computed tomography apparatus may further include an image reconstruction unit that reconstructs a first image of the object by using the first projection data and a second image of the object by using the second projection data.

The image reconstruction unit may reconstruct the first image and the second image of the object by using an iterative algorithm.

According to one or more exemplary embodiments, a method of generating an image by using a computed tomography apparatus includes irradiating an X-ray to an object through a filter unit that comprises a plurality of transmissive areas and a plurality of slightly transmissive areas that are arranged in a predetermined direction, the X-ray being irradiated by an X-ray irradiation unit that rotates around a predetermined rotation path, changing a relative position of the plurality of transmissive areas with respect to the X-ray irradiation unit and a relative position of the plurality of slightly transmissive areas with respect to the X-ray irradiation unit, by controlling a motion of the filter unit, acquiring projection data by detecting an X-ray transmitted through the object, and reconstructing an image of the object by using the projection data.

The acquiring of the projection data may include acquiring first projection data by detecting an X-ray transmitted through the plurality of transmissive areas and second projection data by detecting an X-ray transmitted through the plurality of slightly transmissive areas, and the reconstructing of the image of the object may include reconstructing a first image of the object by using the first projection data and a second image of the object by using the second projection data.

The reconstructing of the first image and the second image may include reconstructing the first image and the second image of the object by using an iterative algorithm.

According to one or more exemplary embodiments, a non-transitory computer-readable storage medium having stored thereon a program, which when executed by a computer, performs the above method.

According to one or more exemplary embodiments, a computed tomography apparatus includes an X-ray irradiation unit irradiating an X-ray to an object while rotating along a predetermined rotation path, a detector acquiring projection data by detecting an X-ray transmitted through the object, a filter unit located between the X-ray irradiation unit and the object and comprising a plurality of transmissive areas and a plurality of non-transmissive areas that are arranged in a predetermined direction, and a control unit controlling a motion of the filter unit to allow a relative position of the plurality of transmissive areas with respect to the X-ray irradiation unit and a relative position of the plurality of non-transmissive areas with respect to the X-ray irradiation unit to change while the X-ray irradiation unit rotates.

The plurality of transmissive areas and the plurality of non-transmissive areas may be alternatingly arranged in the predetermined direction.

A total size of the plurality of transmissive areas may be smaller than a total size of the plurality of non-transmissive areas.

A total size of the plurality of transmissive areas may be ¼ of a total size of the plurality of non-transmissive areas.

The filter unit may include a flat panel in which the plurality of transmissive areas and the plurality of non-transmissive areas are formed in a direction perpendicular to a rotational axis of the X-ray irradiation unit.

The control unit may control a motion of the flat panel to allow the flat panel to perform a reciprocal motion in a direction in which the plurality of transmissive areas and the plurality of non-transmissive areas are formed.

The control unit may control a motion of the flat panel to allow the flat panel to reciprocate 20 times while the X-ray irradiation unit rotates one time.

The filter unit may include an air compressor motor or a vibration motor that transmits a drive force for the reciprocal motion to the flat panel.

The filter unit may include a flat panel in which the plurality of transmissive areas and the plurality of non-transmissive areas are formed in a circular direction.

The control unit may control a motion of the flat panel to allow the flat panel to rotate in the circular direction.

The filter unit may include a caterpillar panel in which the plurality of transmissive areas and the plurality of non-transmissive areas are formed in a direction perpendicular to a rotational axis of the X-ray irradiation unit, and a plurality of driving rollers contacting an inner circumference of the caterpillar panel.

The control unit may control motions of the plurality of driving rollers to allow the caterpillar panel to move in a direction in which the plurality of transmissive areas and the plurality of non-transmissive areas are formed.

The filter unit may include an X-ray non-transmitting member having a spiral shape, a predetermined thickness, and a predetermined pitch interval in which the plurality of transmissive areas and the plurality of non-transmissive areas are formed in a direction perpendicular to a rotational axis of the X-ray irradiation unit.

The control unit may control a motion of the X-ray non-transmitting member to allow the X-ray non-transmitting member to rotate around an axis in a direction in which the plurality of transmissive areas and the plurality of non-transmissive areas are formed.

The detector may acquire projection data by detecting an X-ray transmitted through the plurality of transmissive areas, and the computed tomography apparatus may further include an image reconstruction unit that reconstructs an image of the object by using the projection data.

The image reconstruction unit may reconstruct the image of the object by using an iterative algorithm.

According to one or more exemplary embodiments, a method of generating an image by using a computed tomography apparatus includes irradiating an X-ray to an object through a filter unit that comprises a plurality of transmissive areas and a plurality of non-transmissive areas that are arranged in a predetermined direction, the X-ray being irradiated by an X-ray irradiation unit that rotates around a predetermined rotation path, changing a relative position of the plurality of transmissive areas with respect to the X-ray irradiation unit and a relative position of the plurality of non-transmissive areas with respect to the X-ray irradiation unit, by controlling a motion of the filter unit, acquiring projection data by detecting an X-ray transmitted through the object, and reconstructing an image of the object by using the projection data.

The reconstructing of the image may include reconstructing the image of the object by using an iterative algorithm.

According to one or more exemplary embodiments, a non-transitory computer-readable storage medium having stored thereon a program, which when executed by a computer, performs the above method.

In an exemplary embodiment, there is a computed tomography apparatus including an X-ray irradiator configured to irradiate an X-ray to an object while moving along a curved path, a detector configured to acquire projection data by detecting the X-ray transmitted through the object, a filter disposed between the X-ray irradiator and the object, the filter including a plurality of first transmissive areas and a plurality of second transmissive areas, the plurality of first transmissive areas and the plurality of second transmissive areas being arranged in a predetermined direction; and a controller configured to control a motion of the filter so that a relative position of the plurality of first transmissive areas with respect to the X-ray irradiator and a relative position of the plurality of second transmissive areas with respect to the X-ray irradiator changes while the X-ray irradiator moves along the curved path.

In another exemplary embodiment, there is a method of generating an image by using a computed tomography apparatus, the method including: irradiating an X-ray to an object through a filter that includes a plurality of first transmissive areas and a plurality of second transmissive areas that are arranged in a predetermined direction, the X-ray being output by an X-ray irradiator that moves along a curved path; changing a relative position of the plurality of first transmissive areas with respect to the X-ray irradiator and a relative position of the plurality of second transmissive areas with respect to the X-ray irradiator, by controlling a motion of the filter; acquiring projection data by detecting the X-ray transmitted through the object; and reconstructing an image of the object based on the projection data.

In an exemplary embodiment, there is a computed tomography apparatus including: an X-ray irradiator configured to irradiate an X-ray to an object while moving along a curved path; a detector configured to acquire projection data by detecting an X-ray transmitted through the object; a filter disposed between the X-ray irradiator and the object, the filter including a plurality of transmissive areas and a plurality of non-transmissive areas that are arranged in a predetermined direction; and a controller configured to control a motion of the filter so that a relative position of the plurality of transmissive areas with respect to the X-ray irradiator and a relative position of the plurality of non-transmissive areas with respect to the X-ray irradiator changes while the X-ray irradiator moves along the curved path.

In yet another exemplary embodiment, there is a method of generating an image based on a computed tomography apparatus, the method including: irradiating an X-ray to an object through a filter that includes a plurality of transmissive areas and a plurality of non-transmissive areas that are arranged in a predetermined direction, the X-ray being output by an X-ray irradiator that moves along a circular path; changing a relative position of the plurality of transmissive areas with respect to the X-ray irradiator and a relative position of the plurality of non-transmissive areas with respect to the X-ray irradiator, by controlling a motion of the filter; acquiring projection data by detecting the X-ray transmitted through the object; and reconstructing an image of the object based on the projection data.

In yet another exemplary embodiment, there is a computed tomography apparatus including: an X-ray irradiator configured to irradiate an X-ray along an irradiation direction, to an object while moving along a circular path; a detector configured to acquire projection data by detecting the X-ray transmitted through the object; a filter including a first opening and a second opening, the filter being disposed between the X-ray irradiator and the object, the first opening having a first transmissive property and the second opening having a second transmissive property that is less transmissive than the first transmissive property, and one of the first and the second openings being disposed in the irradiation direction; and a controller configured to move the first and the second openings so that the one of the first and the second openings is moved out of the irradiation direction and another of the first and the second openings is moved into the irradiation direction, during one revolution of the X-ray irradiator along the circular path.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 illustrates a computed tomography (CT) apparatus according to an exemplary embodiment;

FIG. 2A is a graph showing a relationship between the number of photons of an X-ray transmitted through transmissive areas included in a filter unit and X-ray photon energy;

FIG. 2B is a graph showing a relationship between the number of photons of an X-ray transmitted through slightly transmissive areas included in the filter unit and X-ray photon energy;

FIGS. 3A, 3B, and 3C are perspective views illustrating exemplary structures of a filter unit of FIG. 1;

FIGS. 4A and 4B are views for explaining exemplary methods of transmitting a drive force to the flat panel of FIG. 3A;

FIGS. 5A, 5B, and 5C are signograms respectively corresponding to projection data acquired by an X-ray transmitted through the filter unit of FIG. 3A, projection data acquired by an X-ray transmitted through the filter unit of FIG. 3B, and projection data acquired by an X-ray transmitted through the filter unit of FIG. 3C;

FIG. 6 is a flowchart of a method of generating an image by using a CT apparatus, according to an exemplary embodiment;

FIGS. 7A, 7B, 7C, and 7D are exemplary structures of the filter unit of FIG. 1;

FIG. 8 is a signogram corresponding to projection data acquired by an X-ray transmitted through the filter unit of FIG. 7D;

FIG. 9A is a signogram corresponding to full projection data;

FIG. 9B is a signogram corresponding to conventional sparse projection data;

FIG. 9C is a signogram corresponding to sparse projection data acquired by an X-ray transmitted through a fixed filter unit;

FIG. 9D is a signogram corresponding to sparse projection data acquired by an X-ray transmitted through the filter unit of the CT apparatus according to the present exemplary embodiment;

FIG. 10A illustrates an image reconstructed from the full projection data of FIG. 9A;

FIG. 10B illustrates an image reconstructed from the conventional sparse projection data of FIG. 9B;

FIG. 10C illustrates an image reconstructed from the sparse projection data of FIG. 10A;

FIG. 10D illustrates an image reconstructed from the sparse projection data of FIG. 9D;

FIG. 11 is a block diagram schematically illustrating a structure of a CT apparatus according to an exemplary embodiment; and

FIG. 12 is a block diagram schematically illustrating a communication unit of FIG. 11.

### DETAILED DESCRIPTION OF EXEMPLARY EMBODIMENTS

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. As used herein, expressions such as “at least one of,” when preceding a list of elements, modify the entire list of elements and do not modify the individual elements of the list.

The terms used in the present specification are briefly described and the present invention is described in detail.

The terms used in the present invention are those selected from currently widely used general terms in consideration of functions in the present invention. However, the terms may vary according to an engineer's intension, precedents, or advent of new technology. Also, for special cases, terms selected by the applicant are used, in which meanings the selected terms are described in detail in the description section. Accordingly, the terms used in the present invention are defined based on the meanings of the terms and the contents discussed throughout the specification, not by simple meanings thereof.

When a part may “include” a certain constituent element, unless specified otherwise, it may not be construed to exclude another constituent element but may be construed to further include other constituent elements. The terms such as “—portion”, “—unit”, “—module”, and “—block” stated in the specification may signify a unit to process at least one function or operation and the unit may be embodied by hardware such as FPGA or ASIC, software, or a combination of hardware and software. However, the unit may be configured to be located in a storage medium to be addressed or configured to be able to operate one or more processors. Accordingly, the unit as an example includes constituent elements such as software constituent elements, object-oriented software constituent elements, class constituent elements, and task constituent elements, processes, functions, attributes, procedures, sub-routines, segments of program codes, drivers, firmware, microcodes, circuits, data, a database, data structures, tables, arrays, and variables. The constituent elements and functions provided by the “units” may be combined into less number of constituent elements and units or may be further divided into additional constituents and units. Accordingly, the present invention is not limited by a specific combination of hardware and software.

In the present specification, an “image” may signify multi-dimensional data formed of discrete image elements, for example, pixels in a two-dimensional image and voxels in a three-dimensional image. For example, an image may include an X-ray, a computed tomography (CT), a magnetic resonance imaging (MRI), an ultrasound, and a medical image of an object acquired by other medical imaging apparatus.

Also, in the present specification, an “object” may include a human, an animal, or a part of a human or an animal. For example, an object may include organs such as the liver, the heart, the womb, the brain, a breast, the abdomen, etc., or blood vessels. Also, an object may include a phantom that signifies matter having a volume that is approximately the intensity and effective atomic number of a living thing, and may include a sphere phantom having a property similar to a human body.

Also, in the present specification, a “user” may be a doctor, a nurse, a clinical pathologist, a medical imaging expert, a technician who fixes a medical apparatus, etc, but the present exemplary embodiment is not limited thereto.

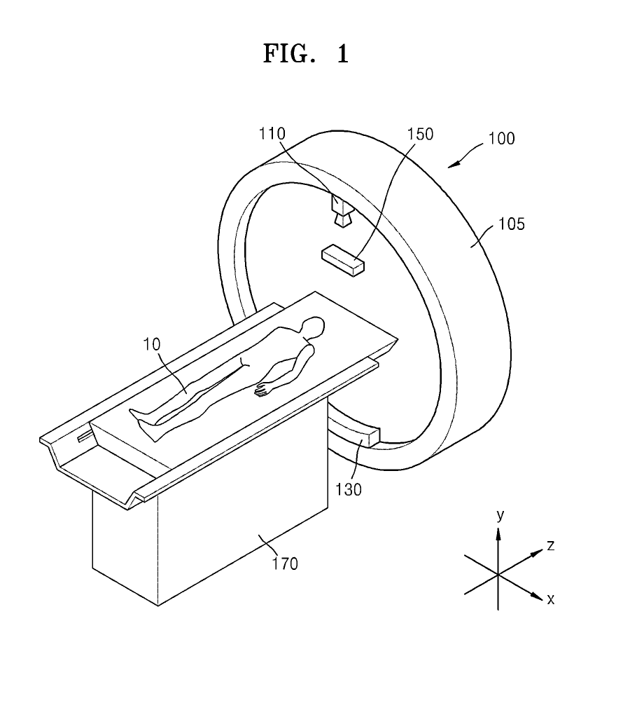

FIG. 1 illustrates a CT apparatus 100 according to an exemplary embodiment. Referring to FIG. 1, the CT apparatus 100 includes an X-ray irradiation unit 110, e.g., an X-ray irradiator, which is attached to a rotating frame 105. The rotating frame 105 rotates about a rotational axis, and hence, the X-ray irradiation unit 110 moves along a predetermined path and irradiates an X-ray to an object 10 (such as a patient). Although X-ray is mentioned in the singular for the sake of brevity, an X-ray as mentioned in the disclosure may include multiple X-rays, or may be an X-ray beam which includes multiple X-rays. There is a detector 130 detecting the X-ray transmitted to the object 10 and acquiring projection data, a filter unit 150, e.g., a filter, located between the X-ray irradiation unit 110 and the object 10 and filtering the X-ray output or irradiated by the X-ray irradiation unit 110, and a control unit, e.g., a controller, controlling a motion of the filter unit 150. In an exemplary embodiment, the X-ray irradiation unit 110 moves along a curved path, e.g., a circular path about an axis that is at the center of the circular path. The axis at the center of the circular path may be coaxial with the rotational axis of the rotating frame 105. The X-ray irradiation unit 110 and the detector 130 may be arranged to face each other with the object 10 interposed therebetween. The control unit may be configured by a microprocessor. Although FIG. 1 illustrates that the filter unit 150 is located outside the X-ray irradiation unit 110, the filter unit 150 may be located within the X-ray irradiation unit 110.

Also, the CT apparatus 100 may further include a table 170 supporting the object 10, and a rotating frame 105 rotating about a Z-axis to allow the detector 130 to move along a path. In an exemplary embodiment, the detector 130 moves along a curved path that may be a circular path about a rotational axis such as the Z-axis. The object 10 may be included in a gantry (not shown) of the CT apparatus 100.

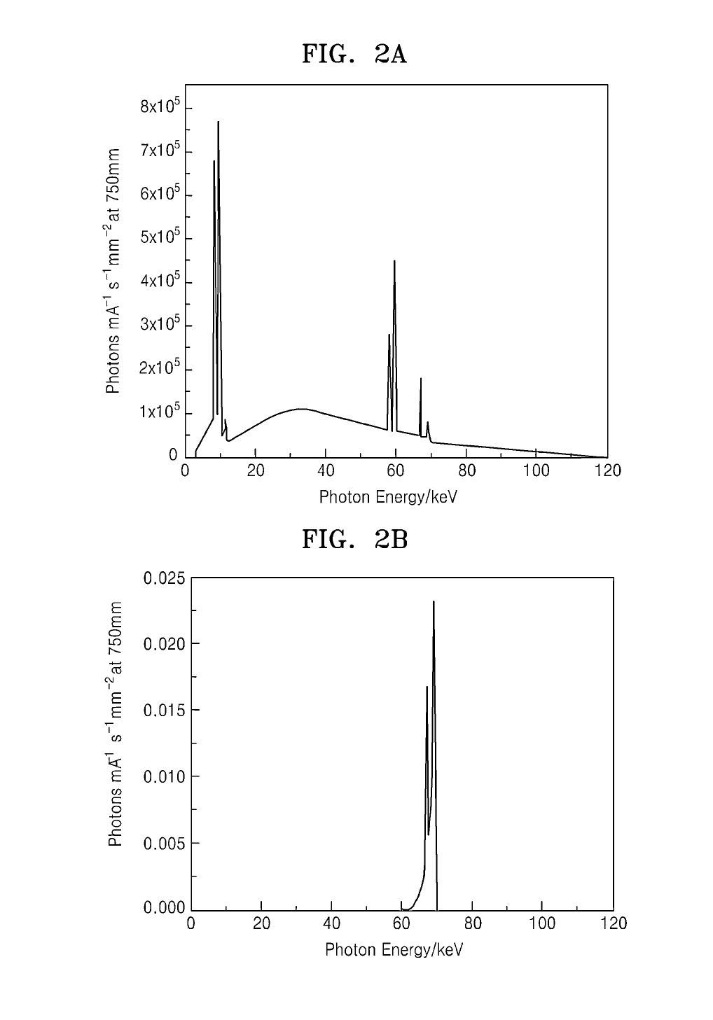

The filter unit 150 includes a plurality of transmissive areas and a plurality of slightly transmissive areas which are arranged along a preset direction for filtering an X-ray. In an exemplary embodiment, the plurality of transmissive areas may be a first plurality of areas and the plurality of slightly transmissive areas may be a second plurality of areas. The transmissive areas and slightly transmissive areas may be alternatingly arranged. The structure of the filter unit 150 will be described later with reference to FIGS. 2A, 2B, and 2C.

The transmissive areas may attenuate the X-ray irradiated by the X-ray irradiation unit 110 by a first attenuation rate. The slightly transmissive areas may attenuate the X-ray irradiated by the X-ray irradiation unit 110 by a second attenuation rate that is greater than the first attenuation rate.

When an X-ray is transmitted through a material having a predetermined thickness, of low-energy photons are absorbed by the material. Accordingly, after being transmitted through the material, the X-ray is configured with high-energy photons so as to have a qualitative change of having an increased transmissivity, which is referred to as a beam hardening effect.

Thus, the X-ray transmitting through the transmissive areas having the first attenuation rate is changed to an X-ray having a low average energy, compared to the X-ray that transmitted through the slightly transmissive areas having the second attenuation rate.

The detector 130 of FIG. 1 may acquire first projection data in a low energy band by detecting the X-ray transmitted through the transmissive areas and second projection data in a high energy band by detecting the X-ray transmitted through the slightly transmissive areas.

The second attenuation rate of the slightly transmissive areas may be set to distinguish the first projection data of a low energy band and the second projection data of a high energy band and also to be able to provide projection information about the object 10. For example, the second attenuation rate may be set to 50% to 95% of the first attenuation rate.

Also, the total size of the transmissive areas may be set to be smaller than the total size of the slightly transmissive areas. It is clear that, as the size of the transmissive areas decrease, a radiation exposure dose on the object 10 decreases. Also, as described above, the strength of the X-ray transmitted through the slightly transmissive areas having the second attenuation rate decreases, which may deteriorate the quality of an image of the object 10. Thus, by increasing the size of the slightly transmissive areas, a redundancy of the projection data according to the movement of the X-ray irradiation unit 110 may be increased and thus noise of the object 10 may be reduced.

Although it is not illustrated in FIG. 1, the CT apparatus 100 according to the present exemplary embodiment may further include an image reconstruction unit, e.g., image reconstructor, that reconstructs a first image of a low energy band from the first projection data of a low energy band and a second image of a high energy band from the second projection data of a high energy band. The image reconstruction unit may reconstruct the first and second images of the object 10 by using an iterative algorithm.

The iterative algorithm may be used to reconstruct an image of the object 10 from sparse projection data. The sparse projection may signify acquiring projection data at a frame rate that is lower than a predetermined frame rate when projection data is generated by using only some of the total detector elements included in the detector 130 or is acquired at a predetermined frame rate by the CT apparatus 100 while the X-ray irradiation unit 110 revolves one time, i.e., while the rotating frame 105 rotates one time. In other words, according to the sparse projection, a small amount of projection data may be acquired compared to full projection in which projection data is generated by using the total detector elements included in the detector 130, and the CT apparatus 100 acquires the projection data at the predetermined frame rate.

The iterative algorithm may more accurately reconstruct an image than a filtered back projection (FBP) algorithm that reconstructs the image of the object 10 from the projection data through a single reconstruction step. The iterative algorithm may include algebraic reconstruction technique (ART), simultaneous iterative reconstruction technique (SIRT), iterative least-squares technique (ILST), a gradient and conjugate gradient (CG) algorithm, maximum likelihood expectation maximization (MLEM), ordered-subsets expectation maximization (OSEM), a maximum a posteriori (MAP) algorithm, or a total variation minimization algorithm.

The image reconstruction unit according to the present exemplary embodiment may reconstruct the first image and the second image by using a total variation minimization algorithm. The total variation minimization algorithm may reduce the number of unknowns of equations or measurements of a given system by using sparsity of strength of an image derivative.

A conventional CT apparatus includes two X-ray irradiation units for irradiating X-rays of different energies and two detectors for detecting the X-rays irradiated by the two X-ray irradiation units, or adopts a method of scanning the object 10 with an X-ray of high energy by using one X-ray irradiation unit and scanning the object 10 again with an X-ray of low energy, for dual energy scan of the object 10. However, the conventional CT apparatus for dual energy scan has a problem of a high radiation exposure dose that is put on the object 10.

The CT apparatus 100 according to the present exemplary embodiment may acquire both the first image of a low energy band and the second image of a high energy band through one-time scanning by the X-ray irradiation unit 110 only and thus the radiation exposure dose on the object 10 may be reduced. Also, since the filter unit 150 only is provided in a general CT apparatus including the X-ray irradiation unit 110 and the detector 130 only, dual energy scan is made possible.

The control unit controls a motion of the filter unit 150 such that a relative position of the transmissive areas with respect to the X-ray irradiation unit 110 and a relative position of the slightly transmissive areas with respect to the X-ray irradiation unit 110 may be changed during the movement of the X-ray irradiation unit 110. As the control unit controls the filter unit 150 to perform a reciprocating motion, a rotational motion or a spinning motion, or a linear motion, the relative position of the transmissive areas with respect to the X-ray irradiation unit 110 and the relative position of the slightly transmissive areas with respect to the X-ray irradiation unit 110 may be changed.

If the filter unit 150 is fixed, the detector elements that detect the X-rays transmitted through the transmissive areas among the detector elements located on the X-axis of FIG. 1 detect only the X-rays transmitted through the transmissive areas while the X-ray irradiation unit 110 revolves 360°. Also, the detector elements that detect the X-rays transmitted through the slightly transmissive areas among the detector elements located on the X-axis of FIG. 1 detect only the X-rays transmitted through the slightly transmissive areas while the X-ray irradiation unit 110 revolves 360°. In other words, projection data sampling is performed irregularly.

The control unit of the CT apparatus 100 according to the present exemplary embodiment may improve uniformity in the projection data sampling by controlling the motion of the filter unit 150.

FIG. 2A is a graph showing a relationship between the number of photons of an X-ray transmitted through the transmissive areas included in the filter unit 150 and X-ray photon energy. FIG. 2B is a graph showing a relationship between the number of photons of an X-ray transmitted through the slightly transmissive areas included in the filter unit 150 and X-ray photon energy.

Referring to FIGS. 2A and 2B, it can be seen that the X-ray transmitted through the transmissive areas includes many photons of an X-ray of a low energy band and the X-ray transmitted through the slightly transmissive areas includes many photons of an X-ray of a high energy band.

Accordingly, the CT apparatus 100 according to the present exemplary embodiment may reconstruct the first image of a low energy band by using an X-ray transmitted through the transmissive areas and having a low average energy and the second image of a high energy band by using an X-ray transmitted through the slightly transmissive areas and having a high average energy.

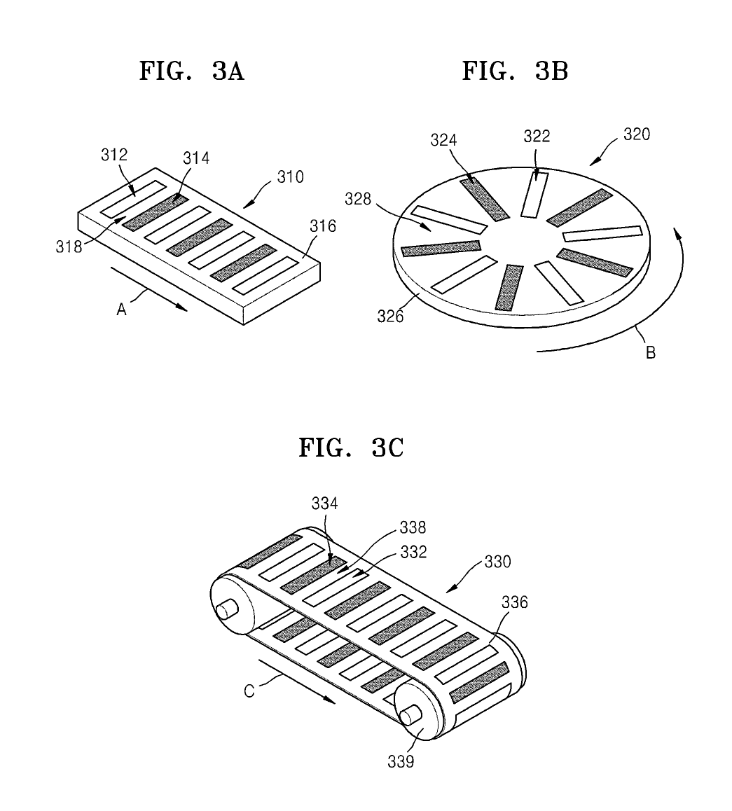

An exemplary structure of the filter unit 150 of the CT apparatus 100 according to the present exemplary embodiment is described below with reference to FIGS. 3A, 3B, and 3C.

FIGS. 3A, 3B, and 3C illustrate exemplary structures of the filter unit 150. Referring to FIG. 3A, a filter unit 310 may include a flat panel 316 in which a plurality of transmissive areas 312, e.g., a plurality of first transmissive areas, and a plurality of slightly transmissive areas 314, e.g., a plurality of second transmissive areas, are formed along a direction perpendicular to the rotational axis of the rotating frame 105 to which the X-ray irradiation unit 110 is attached. When the X-ray irradiation unit 110 is located at the 12 o'clock direction of the object 10, the direction perpendicular to the rotational axis of the rotating frame 105 may correspond to the X-axis direction of FIG. 1.

The transmissive areas 312 may correspond to openings formed in the flat panel 316. The transmissive areas 312 and the slightly transmissive areas 314 may be alternatingly formed in the flat panel 316 along a direction A or a direction opposite to the direction A.

The flat panel 316 may be formed of an X-ray non-transmitting material. When the transmissive areas 312 and the slightly transmissive areas 314 are formed at a predetermined interval, an area between the transmissive areas 312 and the slightly transmissive areas 314, which are alternatingly arranged, may form a non-transmissive area 318. As the X-ray irradiated by the X-ray irradiation unit 110 is blocked by the non-transmissive area 318, the radiation exposure dose on the object 10 may be further reduced.

The control unit controls the motion of the flat panel 316 illustrated in FIG. 3A so that the flat panel 316 may perform a reciprocating motion along the direction A and the opposite direction to the direction A. In other words, as the control unit controls the flat panel 316 to perform a reciprocating motion along the direction A and the opposite direction to the direction A, a relative position between the X-ray irradiation unit 110 and the transmissive areas 312 and a relative position between the X-ray irradiation unit 110 and the slightly transmissive areas 314 may be changed. To enhance uniformity of the projection data sampling, the control unit may control the motion of the flat panel 316 to reciprocate 10 to 100 times while the X-ray irradiation unit 110 revolves one time, i.e., the rotating frame 105 rotates one time. In an exemplary embodiment, the flat panel 316 moves back and forth in a sliding motion during the reciprocating motion.

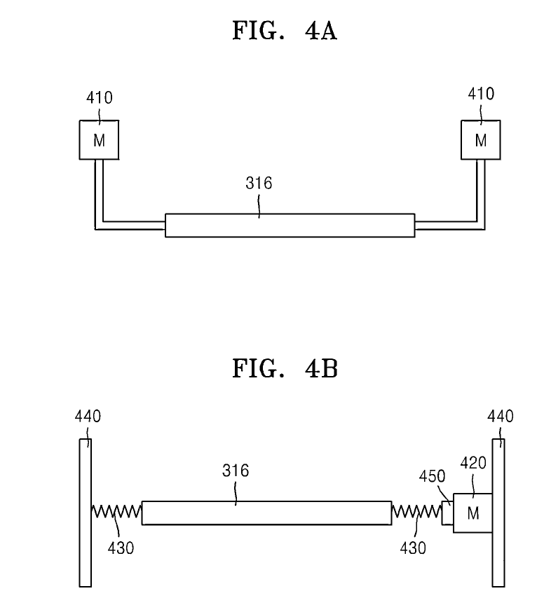

FIGS. 4A and 4B are views for explaining exemplary methods of transmitting a drive force to the flat panel 316 of FIG. 3A. Referring to FIG. 4A, an air compressor motor 410 may convert a compression force by compressed air to a rotational force and transmit a linear force converted from the rotational force to the flat panel 316. The two air compressor motors 410 illustrated in FIG. 3A transmit linear forces in different directions to the flat panel 316 so that the flat panel 316 may perform a reciprocating motion.

FIG. 4B illustrates a vibration motor 420 for transferring a drive force for a reciprocating motion to the flat panel 316. Referring to FIG. 4B, the vibration motor 420 attached to a support 440 transfers a vibration force to the flat panel 316 connected by a spring 430 so that the flat panel 316 performs a reciprocating motion at a predetermined resonance frequency. When a vibration force is continuously supplied to the flat panel 316 that performs a reciprocating motion at a resonant frequency, the dynamic amplitude of the flat panel 316 may infinitely increase according to a resonance phenomenon. Accordingly, a damper 450 for restricting the dynamic amplitude of the flat panel 316 may be connected to the vibration motor 420.

Although only the air compressor motor 410 and the vibration motor 420 that transfers a drive force to the flat panel 316 are described in the present exemplary embodiment, a variety of methods for transferring a drive force for a reciprocating motion to the flat panel 316 may be adopted.

Next, referring to FIG. 3B, a filter unit 320 may include a flat panel 326 in which a plurality of transmissive areas 322 and a plurality of slightly transmissive areas 324 are formed in a radial direction. Referring to FIG. 3B, the transmissive areas 322 and the slightly transmissive areas 324 may be alternatingly formed on the flat panel 326 in a direction B or a direction opposite to the direction B.

The flat panel 326 may be formed of an X-ray non-transmitting material. When the transmissive areas 322 and the slightly transmissive areas 324 are formed to have a predetermined interval, an area between the transmissive areas 322 and the slightly transmissive areas 324, which are alternatingly arranged, may form a non-transmissive area 328.

The control unit controls the motion of the flat panel 326 of FIG. 3B to perform a rotational motion or a spinning motion of the flat panel 326 in the direction B or a direction opposite to the direction B. In other words, as the control unit controls the flat panel 326 to perform a rotational motion in the direction B or a direction opposite to the direction B, a relative position between the X-ray irradiation unit 110 and the transmissive areas 322 and a relative position between the X-ray irradiation unit 110 and the slightly transmissive areas 324 may be changed. To enhance uniformity of the projection data sampling, the control unit may control the motion of the flat panel 326 to reciprocate 1 to 5 times while the X-ray irradiation unit 110 revolves one time, i.e., the rotating frame 105 rotates one time.

Next, referring to FIG. 3C, a filter unit 330 may include a caterpillar panel 336 in which a plurality of transmissive areas 332 and a plurality of slightly transmissive areas 334 are formed in a direction perpendicular to the rotational axis of the rotating frame 105 and a plurality of driving rollers 339 contacting an inner circumference or inner surface of the caterpillar panel 336. When the X-ray irradiation unit 110 is located at the 12 o'clock direction of the object 10, the direction perpendicular to the rotational axis of the rotating frame 105 may correspond to the X-axis direction of FIG. 1.

Referring to FIG. 3C, the transmissive areas 332 and the slightly transmissive areas 334 may be alternatingly formed on the caterpillar panel 336 in a direction C or a direction opposite to the direction C.

The caterpillar panel 336 may be formed of an X-ray non-transmitting material. When the transmissive areas 332 and the slightly transmissive areas 334 are formed to have a predetermined interval, an area between the transmissive areas 332 and the slightly transmissive areas 334, which are alternatingly arranged, may form a non-transmissive area 338.

The control unit controls the driving rollers 339 of FIG. 3C to rotate so that the caterpillar panel 336 may perform a linear motion in the direction C or a direction opposite to the direction C. In other words, as the control unit controls the caterpillar panel 336 to perform a reciprocating motion in the direction C or a direction opposite to the direction C, a relative position between the X-ray irradiation unit 110 and the transmissive areas 332 and a relative position between the X-ray irradiation unit 110 and the slightly transmissive areas 334 may be changed.

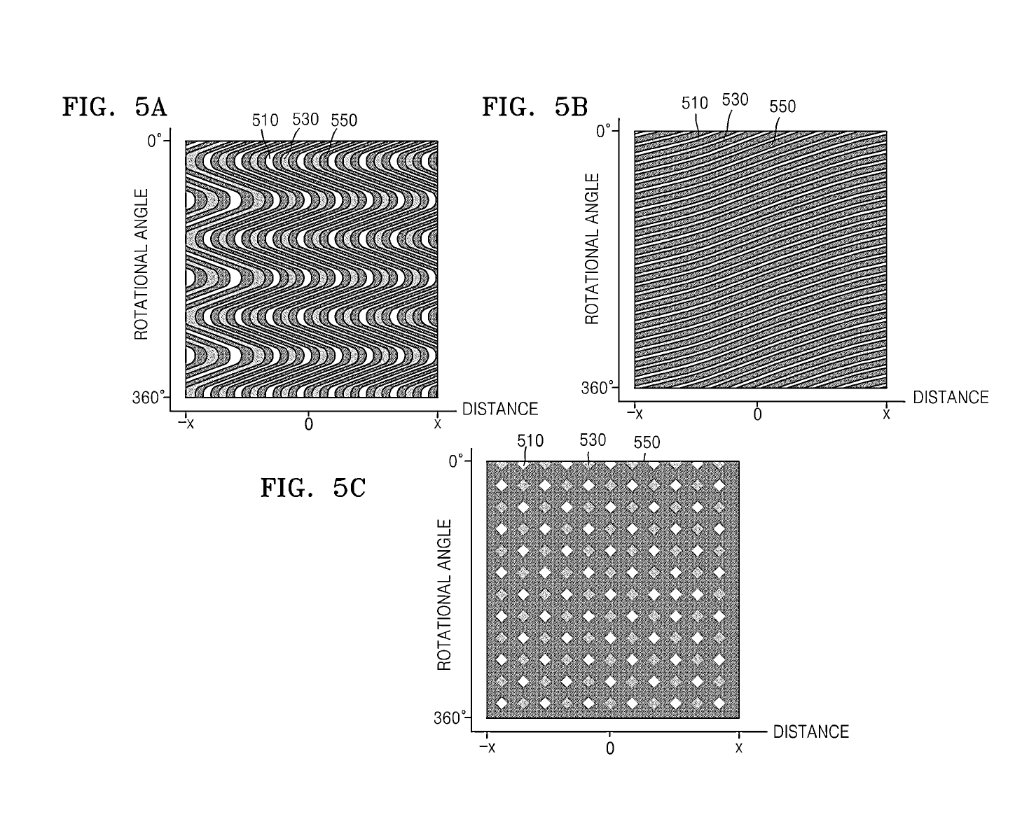

FIGS. 5A, 5B, and 5C are signograms respectively corresponding to projection data acquired by an X-ray transmitted through the filter unit 310 of FIG. 3A, projection data acquired by an X-ray transmitted through the filter unit 320 of FIG. 3B, and projection data acquired by an X-ray transmitted through the filter unit 330 of FIG. 3C.

A signogram is a graph showing projection data acquired by the detector elements located along the X-axis direction perpendicular to the rotational axis of the rotational frame 105 according to the movement of the X-ray irradiation unit 110 when the X-ray irradiation unit 110 revolves 360° and irradiates an X-ray to the object 10.

In the signograms of FIGS. 5A, 5B, and 5C, projection data 510 is acquired by the X-ray transmitted through the transmissive areas 312, 322, and 332 of the filter units 310, 320, and 330; projection data 530 is acquired by the X-ray transmitted through the slightly transmissive areas 314, 324, and 334 of the filter units 310, 320, and 330; and projection data 550 is acquired by the X-ray transmitted through the non-transmissive areas 318, 328, and 338 of the filter units 310, 320, and 330.

If the filter units 310, 320, and 330 of the CT apparatus 100 according to the present exemplary embodiment are fixed, the detector elements for detecting the X-ray transmitted through the non-transmissive areas 318, 328, and 338 of the filter units 310, 320, and 330 may detect only the X-ray transmitted through the non-transmissive areas 318, 328, and 338 of the filter units 310, 320, and 330 while the X-ray irradiation unit 110 moves. Accordingly, projection data sampling may be irregular and thus the quality of an image that is reconstructed may be deteriorated.

Referring to FIGS. 5A, 5B, and 5C, it can be seen that the detector elements located from a position at a −x distance to a position at a +x distance uniformly detect the X-rays transmitted through the transmissive areas 312, 322, and 332, the slightly transmissive areas 314, 324, and 334, and the non-transmissive areas 318, 328, and 338.

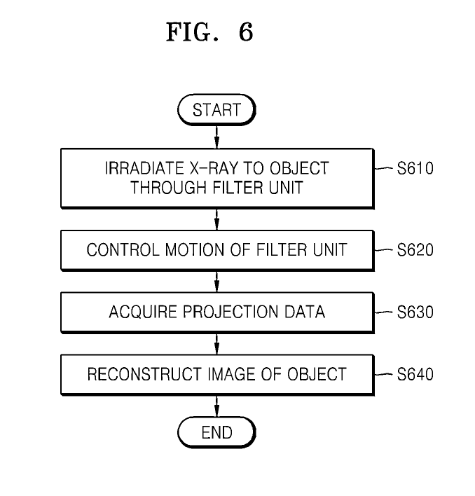

FIG. 6 is a flowchart of a method of generating an image by using a CT apparatus, according to an exemplary embodiment. Referring to FIG. 6, the method of generating an image by using a CT apparatus, according to an exemplary embodiment, includes operation that are time-series processed by the CT apparatus 100 of FIG. 1. Accordingly, although it is omitted in the following description, a description about the CT apparatus 100 of FIG. 1 may be applied to the method of generating an image by using a CT apparatus in FIG. 6.

In S610, the X-ray irradiation unit 110 of the CT apparatus 100 moving along a predetermined path irradiates an X-ray to the object 10 through the filter unit 150 including a plurality of transmissive areas and a plurality of slightly transmissive areas which are arranged in a predetermined direction. In an exemplary embodiment, the path is curved, e.g., circular.

In S620, the CT apparatus 100 controls the motion of the filter unit 150 so that the relative position of the transmissive areas 312, 322, or 332 with respect to the X-ray irradiation unit 110 and the relative position of the slightly transmissive areas 314, 324, or 334 with respect to the X-ray irradiation unit 110 are changed while the X-ray irradiation unit 110 moves.

In S630, the CT apparatus 100 acquires projection data by detecting the X-ray transmitted through the object 10. The CT apparatus 100 may acquire first projection data of a low energy band by detecting the X-ray transmitted through the transmissive areas and second projection data of a high energy band by detecting the X-ray transmitted through the slightly transmissive areas.

In S640, the CT apparatus 100 reconstructs an image of the object 10 by using projection data. The CT apparatus 100 may reconstruct a first image of a low energy band by using the first projection data and a second image of a high energy band by using the second projection data.

The CT apparatus 100 according to another exemplary embodiment may perform low dose radiation scanning on the object 10 by using a filter unit including a plurality of transmissive areas and a plurality of non-transmissive areas only, not the filter unit 150 including the transmissive areas and the slightly transmissive areas. In other words, the image of the object 10 may be reconstructed by using only the projection data acquired by the X-ray transmitted through the transmissive areas. The non-transmissive areas may be formed to have an X-ray attenuation rate so as not to provide projection information about the object 10.

Since the X-ray transmitted through the transmissive areas is incident on some of the total detector elements included in the detector 130, the projection data acquired from the X-ray transmitted through the transmissive areas corresponds to the sparse projection data. Accordingly, the X-ray radiation exposure dose on the object 10 may be reduced.

The transmissive areas and the non-transmissive areas may be alternatingly arranged along a predetermined direction. Also, the total size of the transmissive areas may be smaller than the total size of the non-transmissive areas. Considering the image quality and the X-ray radiation exposure dose on the object 10, the total size of the transmissive areas may be ¼ of the total size of the non-transmissive areas.

The conventional CT apparatus acquires sparse projection data using high speed switching technology of an X-ray irradiation unit. In other words, as a tube current or a tube voltage of the X-ray irradiation unit is switched at a high speed while the X-ray irradiation unit moves, only projection data corresponding to some rotational angles of 0° to 360° is acquired. However, since the technology to switch the tube current or the tube voltage of the X-ray irradiation unit is difficult to implement, the technology is difficult to be applied to a actual clinical CT apparatus.

The CT apparatus 100 according to another exemplary embodiment may acquire sparse projection data by providing only a filter unit in a general CT apparatus.

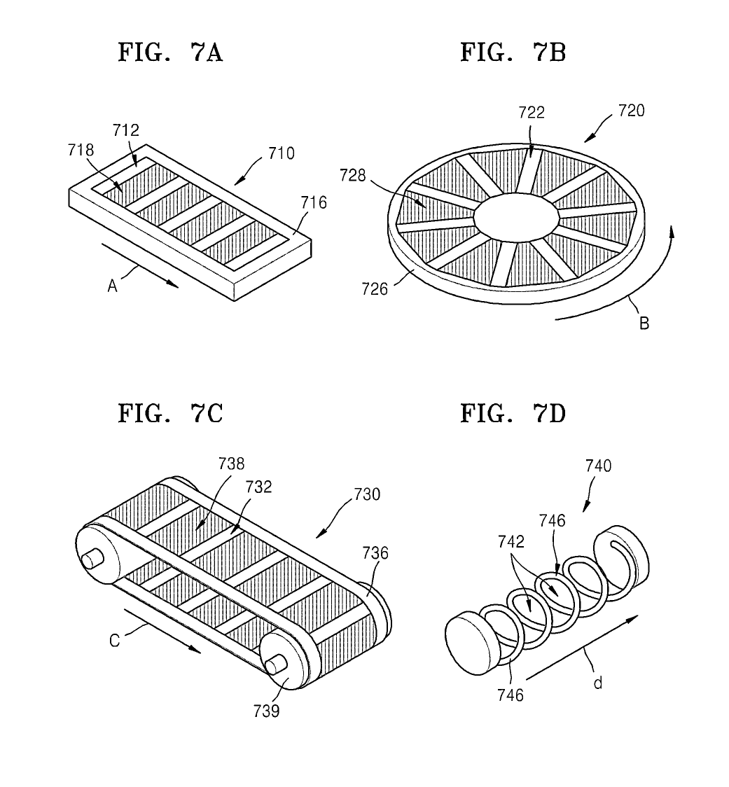

FIGS. 7A, 7B, 7C, and 7D are exemplary structures of the filter unit 150 of FIG. 1. As illustrated in FIG. 7A, a filter unit 710 may include a flat panel 716 in which a plurality of transmissive areas 712 and a plurality of non-transmissive areas 718 are formed in a direction perpendicular to the rotational axis of the rotational frame 105. When the X-ray irradiation unit 110 is located at the 12 o'clock direction of the object 10, the direction perpendicular to the rotational axis may correspond to the direction along the X-axis of FIG. 1.

The transmissive areas 712 may correspond to an opening formed in the flat panel 716. The transmissive areas 712 and the non-transmissive areas 718 may be alternatingly formed on the flat panel 716 in a direction A or a direction opposite to the direction A.

The flat panel 716 may be formed of an X-ray non-transmitting material. When the transmissive areas 712 are formed at a predetermined interval, an area between the transmissive areas 712 may form a non-transmissive area 718.

The control unit controls the motion of the flat panel 716 illustrated in FIG. 7A so that the flat panel 716 may perform a reciprocating motion along the direction A and the opposite direction to the direction A. In other words, as the control unit controls the flat panel 716 to perform a reciprocating motion along the direction A and the opposite direction to the direction A, a relative position between the X-ray irradiation unit 110 and the transmissive areas 712 and a relative position between the X-ray irradiation unit 110 and the non-transmissive areas 718 may be changed. To enhance uniformity of the projection data sampling, the control unit may control the motion of the flat panel 716 to reciprocate about 20 times while the X-ray irradiation unit 110 revolves one time. In an exemplary embodiment, the flat panel 716 moves back and forth in a sliding motion during the reciprocating motion.

The filter unit 710 may include a vibration motor or an air compressor motor for transferring a drive force for a reciprocating motion to the flat panel 716 of FIG. 7A. Since this is already described above with reference to FIGS. 4A and 4B, a detailed description thereof will be omitted herein.

Next, referring to FIG. 7B, a filter unit 720 may include a flat panel 726 in which a plurality of transmissive areas 722 and a plurality of non-transmissive areas 728 are formed in a radial direction. The transmissive areas 722 and the non-transmissive areas 728 may be alternatingly formed on the flat panel 726 in a direction B or a direction opposite to the direction B.

The flat panel 726 may be formed of an X-ray non-transmitting material. When the transmissive areas 722 are formed at a predetermined interval, an area between the transmissive areas 722 may form a non-transmissive area 728.

The control unit controls the motion of the flat panel 726 illustrated in FIG. 7B so that the flat panel 726 may perform a rotational motion or a spinning motion along the direction B and the opposite direction to the direction B. In other words, as the control unit controls the flat panel 726 to perform a rotational motion along the direction B and the opposite direction to the direction B, a relative position between the X-ray irradiation unit 110 and the transmissive areas 722 and a relative position between the X-ray irradiation unit 110 and the non-transmissive areas 728 may be changed.

Next, referring to FIG. 7C, a filter unit 730 may include a caterpillar panel 736 in which a plurality of transmissive areas 732 and a plurality of non-transmissive areas 738 are formed in a direction perpendicular to the rotational axis of the rotating frame 105, and a plurality of driving rollers 739 contacting an inner circumference or an inners surface of the caterpillar panel 736. When the X-ray irradiation unit 110 is located at the 12 o'clock direction of the object 10, the direction perpendicular to the rotational axis may correspond to the direction along the X-axis of FIG. 1.

Referring to FIG. 7C, the transmissive areas 732 and the non-transmissive areas 738 may be alternatingly formed on the caterpillar panel 736 in a direction C or a direction opposite to the direction C.

The caterpillar panel 736 may be formed of an X-ray non-transmitting material. When the transmissive areas 732 are formed at a predetermined interval, an area between the transmissive areas 732 may form a non-transmissive area 738.

The control unit rotates the driving rollers 739 illustrated in FIG. 7C so that the caterpillar panel 736 may perform a motion along the direction C and the opposite direction to the direction C. In other words, as the control unit controls the caterpillar panel 736 to perform a reciprocating motion along the direction C and the opposite direction to the direction C, a relative position between the X-ray irradiation unit 110 and the transmissive areas 732 and a relative position between the X-ray irradiation unit 110 and the non-transmissive areas 738 may be changed.

Next, referring to FIG. 7D, a filter unit 740 may include an X-ray non-transmitting member 746 having a spiral shape, a predetermined thickness, and a predetermined pitch interval, in which a plurality of transmissive areas 742 and a plurality of non-transmissive areas are formed in a direction perpendicular to the rotational axis of the rotating frame 105. When the X-ray irradiation unit 110 is located at the 12 o'clock direction of the object 10, the direction perpendicular to the rotational axis may correspond to the direction along the X-axis of FIG. 1.

Referring to FIG. 7D, the X-ray non-transmitting member 746 forms an X-ray non-transmissive area. A transmissive area 742 is formed in an area where the X-ray non-transmitting member 746 does not exist.

The control unit controls the motion of the X-ray non-transmitting member 746 so that the X-ray non-transmitting member 746 may rotate around a rotational axis in a direction in which the transmissive areas 742 and the non-transmissive areas are formed. In other words, as the control unit controls the X-ray non-transmitting member 746 to perform a rotational motion around the rotational axis in a direction d, a relative position between the X-ray irradiation unit 110 and the transmissive areas 742 and a relative position between the X-ray irradiation unit 110 and the non-transmissive areas may be changed.

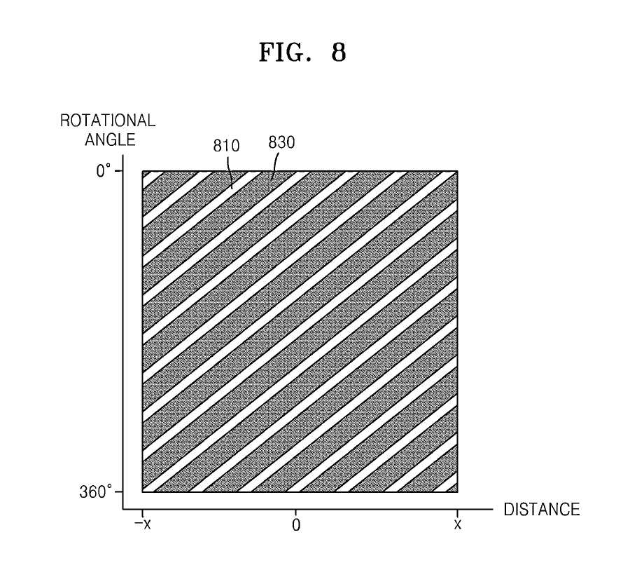

FIG. 8 is a signogram corresponding to projection data acquired by an X-ray transmitted through the filter unit 740 of FIG. 7D.

Signograms acquired by the X-rays transmitted through the filter units 710, 720, and 730 respectively illustrated in FIGS. 7A, 7B, and 7C correspond to the signograms illustrated in FIGS. 5A, 5B, and 5C in which the projection data 530 acquired by the X-ray transmitted through the slightly transmissive areas is changed to the projection data 550 acquired by the X-ray transmitted through the non-transmissive areas.

In FIG. 8, projection data 810 is acquired by the X-ray transmitted through the transmissive areas of the filter unit 740, whereas projection data 830 is acquired by the X-ray transmitted through the non-transmissive areas of the filter unit 740. Referring to FIG. 8, it can be seen that only some of the total detector elements included in the detector 130 may acquire projection data at a certain rotational angle.

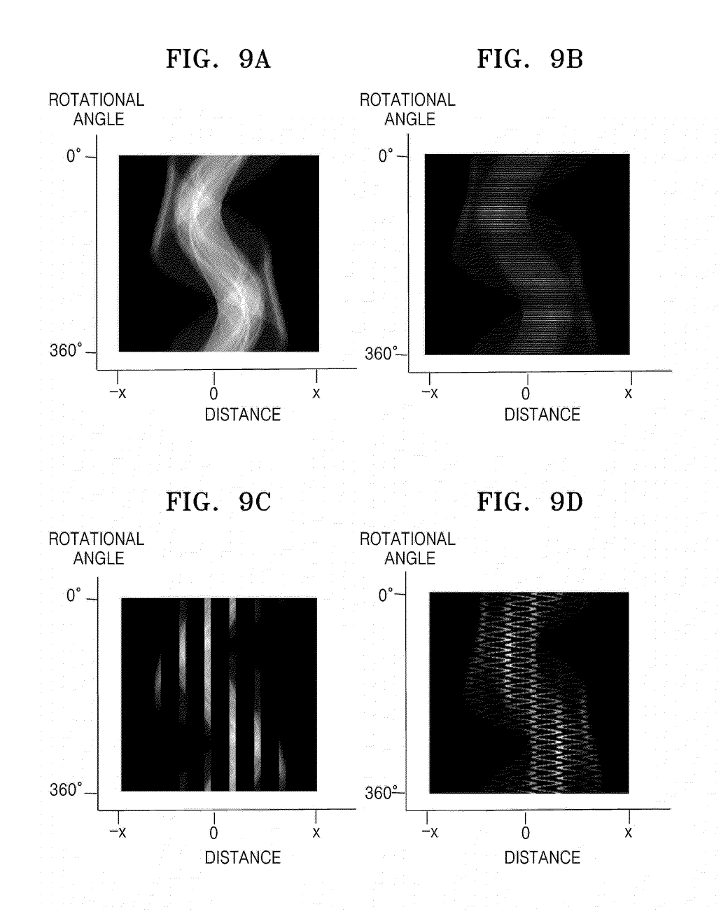

FIG. 9A is a signogram corresponding to full projection data. FIG. 9B is a signogram corresponding to sparse projection data generated according to the conventional high-speed switching technology. FIG. 9C is a signogram corresponding to sparse projection data generated by an X-ray transmitted through a fixed filter unit. FIG. 9D is a signogram corresponding to sparse projection data generated by an X-ray transmitted through the filter unit 150 of the CT apparatus 100 according to the present exemplary embodiment.

The signogram of FIG. 9A is a reference for comparison with the signograms of FIGS. 9B, 9C, and 9D. Referring to FIG. 9C, it can be seen that some of the total detector elements included in the detector 130 do not detect at all the X-ray transmitted through the object 10.

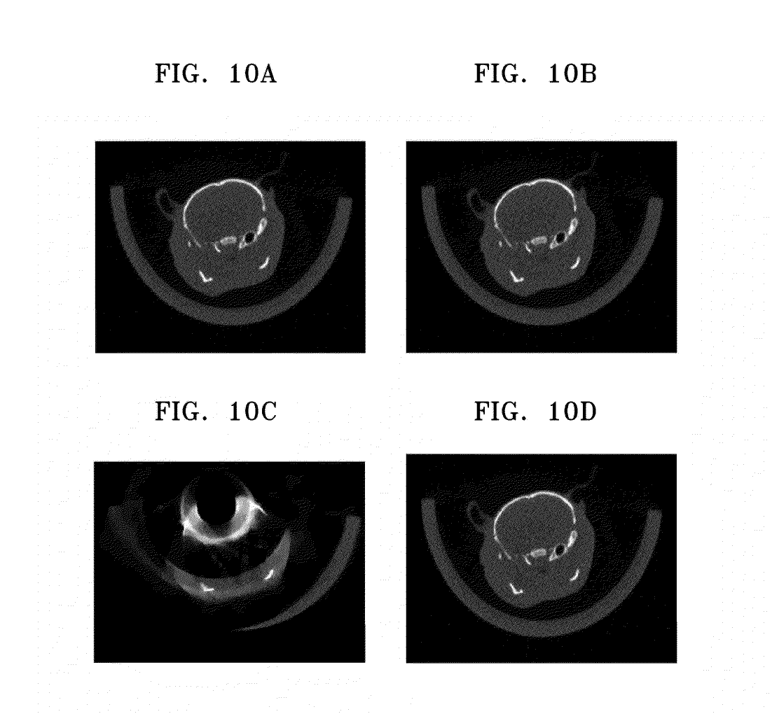

FIG. 10A illustrates an image reconstructed from the full projection data of FIG. 9A. FIG. 10B illustrates an image reconstructed from the sparse projection data of FIG. 9B. FIG. 10C illustrates an image reconstructed from the sparse projection data of FIG. 10A. FIG. 10D illustrates an image reconstructed from the sparse projection data of FIG. 9D.

Referring to FIG. 10C, it can be seen that the quality of a reconstructed image may be much degraded when there are detector elements that do not detect at all the X-ray transmitted through the object 10 among the total detector elements included in the detector 130.

When the image reconstructed from conventional sparse projection data illustrated in FIG. 10B and the image reconstructed according to an exemplary embodiment illustrated in FIG. 10D are compared with the image reconstructed from the full projection data illustrated in FIG. 10A, it can be seen that the quality of the image reconstructed according to an exemplary embodiment is very high.

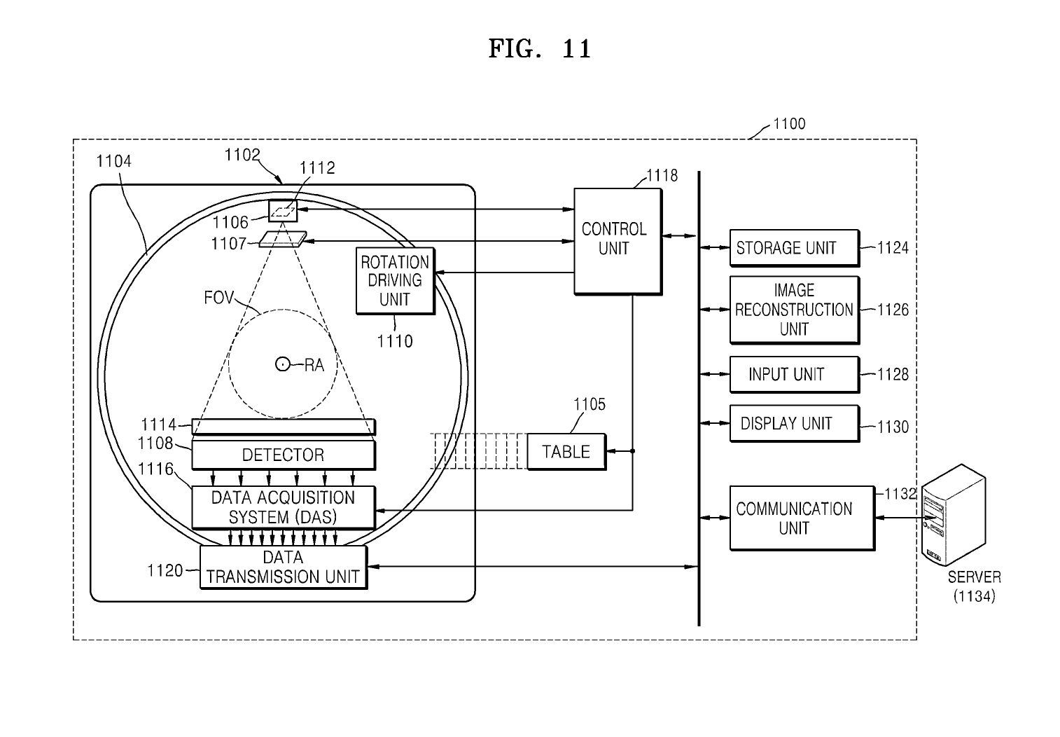

FIG. 11 is a block diagram schematically illustrating a structure of a CT apparatus 1100 according to an exemplary embodiment.

The CT apparatus 1100 according to the present exemplary embodiment may include a gantry 1102, a table 1105, a control unit 1118, a storage unit 1124, an image reconstruction unit 1126, an input unit 1128, a display unit 1130, and a communication unit 1132.

As described above, an object (not shown) may be placed on the table 1105. The table 1105 according to the present exemplary embodiment may be movable in a predetermined direction, for example, at least one of upward, downward, left, and right directions. A motion of the table 1105 may be controlled by the control unit 1118.

The gantry 1102 according to the present exemplary embodiment may include a rotating frame 1104, an X-ray irradiation unit 1106, a filter unit 1107, a detector 1108, a rotation driving unit 1110, a data acquisition system (DAS) 1116, and a data transmission unit 1120.

The rotating frame 1104 of the gantry 1102, according to the present exemplary embodiment, may have a ring shape and be rotatable around a predetermined rotational axis (RA). Also, the rotating frame 1104 may have a disc shape.

The rotating frame 1104 may include the X-ray irradiation unit 1106 and the detector 1108 that are arranged facing each other to have a predetermined field of view (FOV). Also, the rotating frame 1104 may include an anti-scatter grid 1114. The anti-scatter grid 1114 may be located between the X-ray irradiation unit 1106 and the detector 1108.

In a medical imaging system, the X-ray arriving at the detector (or photosensitive film) 1108 may include not only an attenuated primary radiation that forms a useful image but also a scattered radiation that degrades the quality of an image. To transmit most of the primary radiation and attenuate the scattered radiation, the anti-scatter grid 1114 may be located between a patient and the detector 1108.

For example, the anti-scatter grid 1114 may have a form in which strips of lead foil and interspace materials such as solid polymer materials, solid polymers, and fiber composite materials are alternatingly stacked. However, the form of the anti-scatter grid 1114 is not limited thereto.

The filter unit 1107 may include a plurality of transmissive areas (not shown) and a plurality of slightly transmissive areas (not shown) that are arranged in a predetermined direction for filtering an X-ray. Also, the filter unit 1107 may include a plurality of transmissive areas (not shown) and a plurality of non-transmissive areas (not shown) that are arranged in a predetermined direction for filtering an X-ray. The transmissive areas and the slightly transmissive areas, and the transmissive areas and the non-transmissive areas, may be alternatingly arranged with each other.

The rotating frame 1104 may receive a drive signal from the rotation driving unit 1110 and move the X-ray irradiation unit 1106 and the detector 1108 at a predetermined speed. The rotating frame 1104 may receive a drive signal and power from the rotation driving unit 1110 in a contact manner via a slip ring (not shown). Also, the rotating frame 1104 may receive the drive signal and power from the rotation driving unit 1110 via wireless communication.

The X-ray irradiation unit 1106 may receive a voltage and a current from a power distribution unit (PDU) (not shown) via a slip ring (not shown) and a high voltage generation unit (not shown) and may generate and irradiate an X-ray. When the high voltage generation unit applies a predetermined voltage (hereinafter, referred to as the tube voltage) to the X-ray irradiation unit 1106, the X-ray irradiation unit 1106 in response to the predetermined tube voltage may generate X-rays having a plurality of energy spectrums.

The X-ray generated by the X-ray irradiation unit 1106 may be emitted in a predetermined form by a collimator 1112.

The detector 1108 may be arranged to face the X-ray irradiation unit 1106. The detector 1108 may include a plurality of X-ray detection devices. A single X-ray detection device may form a single channel, but the present exemplary embodiment is not limited thereto.

The detector 1108 may detect an X-ray generated by the X-ray irradiation unit 1106 and transmitted through the object and may generate an electric signal corresponding to the strength of a detected X-ray.

The detector 1108 may include an indirect detector that detects an X-ray by converting the X-ray to light and a direct detector that detects an X-ray by converting the X-ray directly to electric charges. The indirect detector may use a scintillator. Also, the direct detector may use a photon counting detector. The DAS 1116 may be connected to the detector 1108. Electric signals generated by the detector 1108 may be collected by the DAS 1116 in a wired or wireless manner. Also, the electric signals generated by the detector 1108 may be provided to an analog-to-digital converter (not shown) via an amplifier (not shown).

Only a part of data collected by the detector 1108 may be provided to the image reconstruction unit 1126 according to the thickness or number of slices. Alternatively, the image reconstruction unit 1126 may select only a part of the data.

A digital signal provided from analog-to-digital converter (not shown) may be provided to the image reconstruction unit 1126 via the data transmission unit 1120. The digital signal may be transmitted to the image reconstruction unit 1126 in a wired or wireless manner via the data transmission unit 1120.

The control unit 1118 according to the present exemplary embodiment may control the operation of each module of the CT apparatus 1100. For example, the control unit 1118 may control operations of the table 1105, the filter unit 1107, the rotation driving unit 1110, the collimator 1112, the DAS 1116, the storage unit 1124, the image reconstruction unit 1126, the input unit 1128, the display unit 1130, the communication unit 1132, etc. In particular, the control unit 1118 may control the motion of the filter unit 1107 such that a relative position of the transmissive areas of the filter unit 1107 with respect to the X-ray irradiation unit 1106 and a relative position of the non-transmissive areas of the filter unit 1107 with respect to the X-ray irradiation unit 1106 can be changed while the X-ray irradiation unit 1106 moves, i.e., while the rotating frame 1104 rotates.

The image reconstruction unit 1126 may receive data acquired by the DAS 1116, for example, pure data before processing, via the data transmission unit 1120 and perform pre-processing.

The pre-processing may include, for example, a process of correcting irregular sensitivity between channels, a process of correcting radial reduction of signal strength or loss of a signal due to an X-ray absorbing member such as metal, etc.

Output data of the image reconstruction unit 1126 may be referred to as raw data or projection data. The projection data may be stored in the storage unit 1124 with photographing conditions such as a tube voltage, a photographing angle, etc, during data acquisition.

The projection data may be a set of data values corresponding to the strength of the X-ray transmitted through the object. For convenience of explanation, a set of projection data simultaneously acquired at the same photographing angle with respect to all channels is referred to as a projection data set.

The storage unit 1124 may include at least one type of storage media such as flash memory, hard disks, a multimedia card, card type memory such as SD memory, XD memory, etc., random access memory (RAM), static random access memory (SRAM), read-only memory (ROM), electrically erasable programmable read-only memory (EEPROM), programmable read-only memory (PROM), magnetic memory, magnetic discs, optical discs, etc.

Also, the image reconstruction unit 1126 may reconstruct a sectional image of the object by using the acquired projection data set. The sectional image maybe a 3-dimensional image. In other words, the image reconstruction unit 1126 may generate a 3-dimensional image of the object by using a cone beam reconstruction method based on the acquired projection data set.

Also, when first projection data corresponding to a low energy band and second projection data corresponding to a high energy band are acquired by the detector 1108, the image reconstruction unit 1126 may reconstruct a first image corresponding to the low energy band from the first projection data and a second image corresponding to the high energy band from the second projection data.

An external input such as an X-ray tomography condition, an image processing condition, etc. may be received through the input unit 1128. For example, the X-ray tomography condition may include tube voltage, setting energy values of a plurality of X-rays, photography protocol selection, image reconstruction method selection, FPV area setting, number of slices, slice thickness, image post-processing parameter setting, etc. Also, the image processing condition may include a resolution of an image, image attenuation coefficient setting, image combination rate setting, etc.

The input unit 1128 may include a device for receiving a predetermined external input. For example, the input unit 1128 may include a microphone, a keyboard, a mouse, a joystick, a touchpad, a touch pen, voice, a gesture recognition apparatus, etc.

The display unit 1130 may display an image reconstructed by the image reconstruction unit 1126.

The transmitting/receiving of data and power between the above-described elements may be performed by using at least one of wired, wireless, and optical communication methods.

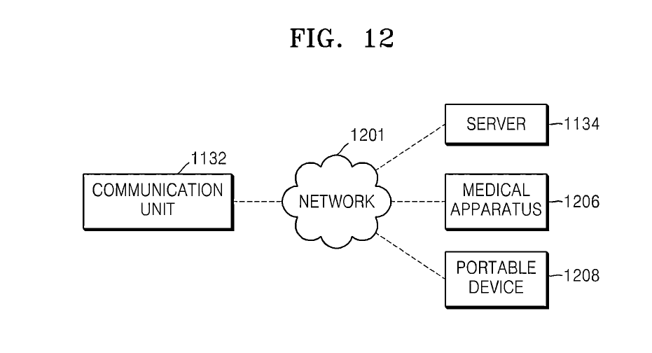

The communication unit 1132 may communicate with an external device, an external medical apparatus, etc. via a server 1134, which will be described later with reference to FIG. 12.

FIG. 12 illustrates the communication unit 1132 of FIG. 11.

The communication unit 1132 is connected to a network 1201 in a wired or wireless manner to communicate with the server 1134, a medical apparatus 1206, or a portable device 1208. The communication unit 1132 may communicate data with the server 1134 or the medical apparatus 1206 in the hospital connected through a picture archiving and communication system (PACS).

The communication unit 1132 may perform data communication with the portable device 1208 according to the digital imaging and communications in medicine (DICOM) standard.

The communication unit 1132 may transmit/receive data related to diagnosis of the object. The communication unit 1132 may transmit/receive a medical image acquired by the medical apparatus 1206 such as an MRI apparatus or an X-ray apparatus.

Furthermore, the communication unit 1132 may receive a patient's diagnostic history or treatment schedule from the server 1134 to be used for clinical diagnosis of a patient. Also, the communication unit 1132 may perform data communication not only with the server 1134 or the medical apparatus 1206 in a hospital but also with the portable device 1208 of a user or patient.

Also, information about a state of equipment and a current status of quality management is transmitted to a system manager or a person in charge of services and a feedback thereof is received, via the network 1201.

The invention can also be embodied as computer-readable codes on a computer-readable recording medium. The computer-readable recording medium is any data storage device that can store data which can be thereafter read by a computer system. Examples of the computer-readable recording medium include ROM, RAM, CD-ROMs, magnetic tapes, floppy disks, optical data storage devices, etc. The computer-readable recording medium can also be distributed over network-coupled computer systems so that the computer-readable code is stored and executed in a distributed fashion

It should be understood that the exemplary embodiments described therein should be considered in a descriptive sense only and not for purposes of limitation. Descriptions of features or aspects within each embodiment should typically be considered as available for other similar features or aspects in other embodiments.

While one or more exemplary embodiments have been described with reference to the figures, it will be understood by those of ordinary skill in the art that various changes in form and details may be made therein without departing from the spirit and scope of the present invention as defined by the following claims.

What is claimed is:

1. A computed tomography apparatus comprising:
an X-ray irradiator configured to irradiate an X-ray to an object while moving along a curved path;
a detector configured to acquire projection data by detecting the X-ray transmitted through the object;
a filter disposed between the X-ray irradiator and the object, the filter comprising a plurality of first transmissive areas and a plurality of second transmissive areas, the plurality of first transmissive areas and the plurality of second transmissive areas being arranged in a predetermined direction; and
a controller configured to control a motion of the filter so that a relative position of the plurality of first transmissive areas with respect to the X-ray irradiator and a relative position of the plurality of second transmissive areas with respect to the X-ray irradiator changes while the X-ray irradiator moves along the curved path.

2. The computed tomography apparatus of claim 1, wherein the plurality of first transmissive areas and the plurality of second transmissive areas are alternatingly arranged in the predetermined direction.

3. The computed tomography apparatus of claim 1, wherein an X-ray attenuation rate of the plurality of first transmissive areas is lower than an X-ray attenuation rate of the plurality of second transmissive areas.

4. The computed tomography apparatus of claim 3, wherein the X-ray attenuation rate of the plurality of first transmissive areas is in a range of 50% to 95% of the X-ray attenuation rate of the plurality of second transmissive areas.

5. The computed tomography apparatus of claim 1, wherein a total size of the plurality of first transmissive areas is smaller than a total size of the plurality of second transmissive areas.

6. The computed tomography apparatus of claim 5, wherein the total size of the plurality of transmissive areas is smaller than ½ of the total size of the plurality of second transmissive areas.

7. The computed tomography apparatus of claim 1, wherein the curved path is a circular path that is about an axis and the filter further comprises a flat panel in which the plurality of first transmissive areas and the plurality of second transmissive areas are formed in a direction perpendicular to the axis of the circular path.

8. The computed tomography apparatus of claim 7, wherein the controller is further configured to control a motion of the flat panel so that the flat panel moves in a reciprocating motion in a direction in which the plurality of first transmissive areas and the plurality of second transmissive areas are formed.

9. The computed tomography apparatus of claim 8, wherein the controller is further configured to control the motion of the flat panel so that the reciprocating motion of the flat panel reciprocates 10 to 100 times while the X-ray irradiator revolves about the axis one time.

10. The computed tomography apparatus of claim 8, wherein the filter further comprises a driving motor that comprises an air compressor motor or a vibration motor, the driving motor being configured to transmit a drive force for the reciprocating motion to the flat panel.

11. The computed tomography apparatus of claim 1, wherein the filter further comprises a flat panel in which the plurality of first transmissive areas and the plurality of second transmissive areas are formed in a circular direction.

12. The computed tomography apparatus of claim 11, wherein the controller is further configured to control a motion of the flat panel so that the flat panel spins.

13. The computed tomography apparatus of claim 12, wherein the curved path is a circular path that is about an axis and wherein the controller is further configured to control the motion of the flat panel so that the flat panel spins one to five times while the X-ray irradiator revolves about the axis of the circular path one time.

14. The computed tomography apparatus of claim 1, wherein the curved path is a circular path that is about an axis and
wherein the filter further comprises:
a caterpillar panel in which the plurality of first transmissive areas and the plurality of second transmissive areas are formed in a direction perpendicular to the axis of the circular path; and
a plurality of driving rollers contacting an inner surface of the caterpillar panel.

15. The computed tomography apparatus of claim 14, wherein the controller is further configured to control motions of the plurality of driving rollers so that the caterpillar panel moves in a direction in which the plurality of first transmissive areas and the plurality of second transmissive areas are formed.

16. The computed tomography apparatus of claim 1, wherein the detector acquires first projection data by detecting the X-ray transmitted through the plurality of first transmissive areas and second projection data by detecting the X-ray transmitted through the plurality of second transmissive areas, and
the computed tomography apparatus further comprises an image reconstructor configured to reconstruct a first image of the object based on the first projection data and a second image of the object based on the second projection data.

17. The computed tomography apparatus of claim 16, wherein the image reconstructor reconstructs the first image and the second image of the object based on an iterative algorithm.

18. A method of generating an image by using a computed tomography apparatus, the method comprising:
irradiating an X-ray to an object through a filter that includes a plurality of first transmissive areas and a plurality of second transmissive areas that are arranged in a predetermined direction, the X-ray being output by an X-ray irradiator that moves along a curved path;
changing a relative position of the plurality of first transmissive areas with respect to the X-ray irradiator and a relative position of the plurality of second transmissive areas with respect to the X-ray irradiator, by controlling a motion of the filter;
acquiring projection data by detecting the X-ray transmitted through the object; and
reconstructing an image of the object based on the projection data.

19. The method of claim 18, wherein the acquiring of the projection data comprises acquiring first projection data by detecting the X-ray transmitted through the plurality of first transmissive areas and second projection data by detecting the X-ray transmitted through the plurality of second transmissive areas, and
the reconstructing of the image of the object comprises reconstructing a first image of the object based on the first projection data and a second image of the object based on the second projection data.

20. The method of claim 19, wherein the reconstructing of the first image of the object and the reconstructing of the second image of the object comprise reconstructing the first image and the second image of the object based on an iterative algorithm.

21. A non-transitory computer-readable storage medium having stored thereon a program, which when executed by a computer, performs the method of claim 18.

22. A computed tomography apparatus comprising:
an X-ray irradiator configured to irradiate an X-ray to an object while moving along a curved path;
a detector configured to acquire projection data by detecting an X-ray transmitted through the object;
a filter disposed between the X-ray irradiator and the object, the filter comprising a plurality of transmissive areas and a plurality of non-transmissive areas that are arranged in a predetermined direction; and
a controller configured to control a motion of the filter so that a relative position of the plurality of transmissive areas with respect to the X-ray irradiator and a relative position of the plurality of non-transmissive areas with respect to the X-ray irradiator changes while the X-ray irradiator moves along the curved path.

23. The computed tomography apparatus of claim 22, wherein the plurality of transmissive areas and the plurality of non-transmissive areas are alternatingly arranged in the predetermined direction.

24. The computed tomography apparatus of claim 22, wherein a total size of the plurality of transmissive areas is smaller than a total size of the plurality of non-transmissive areas.

25. The computed tomography apparatus of claim 24, wherein a total size of the plurality of transmissive areas is ¼ of a total size of the plurality of non-transmissive areas.

26. The computed tomography apparatus of claim 22, wherein the curved path is a circular path this is about an axis and the filter further comprises a flat panel in which the plurality of transmissive areas and the plurality of non-transmissive areas are formed in a direction perpendicular to the axis of the circular path.

27. The computed tomography apparatus of claim 26, wherein the controller is further configured to control a motion of the flat panel so that the flat panel performs a reciprocating motion in a direction in which the plurality of transmissive areas and the plurality of non-transmissive areas are formed.

28. The computed tomography apparatus of claim 27, wherein the controller is further configured to control the motion of the flat panel so that the flat panel to reciprocatingly moves 20 times while the X-ray irradiator revolves about the axis one time.

29. The computed tomography apparatus of claim 27, wherein the filter further comprises a driving motor, the driving motor comprising an air compressor motor or a vibration motor, the driving motor transmitting a drive force for the reciprocating motion to the flat panel.

30. The computed tomography apparatus of claim 22, wherein the filter further comprises a flat panel in which the plurality of transmissive areas and the plurality of non-transmissive areas are formed in a circular direction.

31. The computed tomography apparatus of claim 30, wherein the controller is further configured to control a motion of the flat panel so that the flat panel spins in the circular direction.

32. The computed tomography apparatus of claim 22, wherein the curved paths is a circular path about an axis and wherein the filter further comprises:
a caterpillar panel in which the plurality of transmissive areas and the plurality of non-transmissive areas are formed in a direction perpendicular to the axis circular path; and
a plurality of driving rollers which contact an inner surface of the caterpillar panel.

33. The computed tomography apparatus of claim 32, wherein the controller is further configured to control motions of the plurality of driving rollers so that the caterpillar panel moves in a direction in which the plurality of transmissive areas and the plurality of non-transmissive areas are formed.

34. The computed tomography apparatus of claim 22, wherein the curved paths is a circular path about an axis and wherein the filter comprises an X-ray non-transmitting member having a spiral shape, a predetermined thickness, and a predetermined pitch interval in which the plurality of transmissive areas and the plurality of non-transmissive areas are formed in a direction perpendicular to the axis.

35. The computed tomography apparatus of claim 34, wherein the controller is further configured to control a motion of the X-ray non-transmitting member so that the X-ray non-transmitting member rotates around an axis in a direction in which the plurality of transmissive areas and the plurality of non-transmissive areas are formed.

36. The computed tomography apparatus of claim 22, wherein the detector acquires projection data by detecting the X-ray transmitted through the plurality of transmissive areas, and
the computed tomography apparatus further comprises an image reconstructor that reconstructs an image of the object based on the projection data.

37. The computed tomography apparatus of claim 36, wherein the image reconstructor reconstructs the image of the object based on an iterative algorithm.

38. A method of generating an image based on a computed tomography apparatus, the method comprising:
irradiating an X-ray to an object through a filter that comprises a plurality of transmissive areas and a plurality of non-transmissive areas that are arranged in a predetermined direction, the X-ray being output by an X-ray irradiator that moves along a circular path;
changing a relative position of the plurality of transmissive areas with respect to the X-ray irradiator and a relative position of the plurality of non-transmissive areas with respect to the X-ray irradiator, by controlling a motion of the filter;
acquiring projection data by detecting the X-ray transmitted through the object; and
reconstructing an image of the object based on the projection data.

39. The method of claim 38, wherein the reconstructing of the image comprises reconstructing the image of the object based on an iterative algorithm.

40. A non-transitory computer-readable storage medium having stored thereon a program, which when executed by a computer, performs the method of claim 39.

41. A computed tomography apparatus comprising:
an X-ray irradiator configured to irradiate an X-ray along an irradiation direction, to an object while moving along a circular path;
a detector configured to acquire projection data by detecting the X-ray transmitted through the object;
a filter comprising a first opening and a second opening, the filter being disposed between the X-ray irradiator and the object, the first opening having a first transmissive property and the second opening having a second transmissive property that is less transmissive than the first transmissive property, and one of the first and the second openings being disposed in the irradiation direction; and
a controller configured to move the first and the second openings so that the one of the first and the second openings is moved out of the irradiation direction and another of the first and the second openings is moved into the irradiation direction, during one revolution of the X-ray irradiator along the circular path.

42. The computed tomography apparatus of claim 41, wherein the filter is linearly moved from the one of the first and the second openings to the other of the first and the second openings.

43. The computed tomography apparatus of claim 41, wherein the filter is rotated about a spinning axis.
